(19) United States
(12) Patent Application Publication
(10) Pub. No.: US 2002/0077569 A1
(43) Pub. Date: Jun. 20, 2002

(54) Apparatus and method for measuring the condition of articular cartilage

(76) Inventors: Kyriacos A. Athanasiou, Houston, TX (US); Dan R. Lanctot, San Antonio, TX (US); C. Mauli Agrawal, San Antonio, TX (US)

Correspondence Address: CONLEY ROSE & TAYON, P.C., P. O. BOX 3267, HOUSTON, TX, 77253-3267, US

(21) Appl. No.: 09/995,881

(22) Filed: Nov. 28, 2001

Related U.S. Application Data
(60) Provisional application No. 60/253,466, filed on Nov. 28, 2000.

(51) Int. Cl. A61B 5/103

(52) U.S. Cl. 600/587; 600/553

(57) ABSTRACT

An apparatus 10 has a probe 22 disposed within a housing 12. A distal end 18 of the probe 22 is adapted to contact a surface of the articular cartilage and a proximal end 26 of the probe 22 is disposed within the housing 12. The apparatus 10 also includes a mass 28 disposed within the housing 12 at a position spaced from the proximal end 26 of the probe 22, and an accelerator 30 coupled to the mass 28 and configured to accelerate the mass 28 to a predetermined velocity. The mass 28, when accelerated to the predetermined velocity, creates a force that is applied to the proximal end 26 of the probe 22, thereby transmitting the predetermined force through the distal end 24 of the probe 22 to the cartilage. The mechanical response of the cartilage to the application of the force applied by the distal end 24 of the probe 22 is detected and used to assess the biomechanical condition of the cartilage.

### CROSS-REFERENCE TO RELATED APPLICATIONS

The present application claims benefit oil U.S. provisional application Serial No. 60/253,466, filed Nov. 28, 2000, and entitled “Apparatus And Method For Measuring The Condition Of Articular Cartilage.”

### STATEMENT REGARDING FEDERALLY SPONSORED RESEARCH OR DEVELOPMENT

Not applicable.

### TECHNICAL FIELD OF THE INVENTION

The present invention relates generally to in apparatus and method for evaluating the condition of articular cartilage, and more particularly to an apparatus and method for applying a predetermined post-contact impact load to a probe i a contact with the cartilage being evaluated.

### BACKGROUND OF THE INVENTION

Articular cartilage in diarthrodial joints is subject to deterioration as a function of osteoarthritis, injury, and other disease processes. Several devices and methods previously have been proposed for evaluating the condition of articular cartilage. For example, U.S. Pat. No. 5,003,982 issued to Henry R. Halperin on Apr. 2, 1991 for a Dynamic Indentation System describes a system which uses cyclical probe indentation to determine mechanical properties, such as in-plane wall stress, of living tissue, e.g., the heart muscle. In the Halperin device, the probe moves in a linear fashion, vibrating at a frequency of-about 320 Hz, through an amplitude of from about 0.1 to about 0.5 mm. The indentation pressure force is measured several times during the cyclic movement of the probe.

More recently, U.S. Pat. Nos. 5,433,215; 5,503,162; and 5,673,708 issued to Kyriacos A. Athanasiou, et al., for arthroscopic cartilage evaluators that make use of a linear actuator (motor) to move a probe tip toward a tissue surface,. It should be noted that Dr. Athanasiou is a co-inventor of the present invention. The previous Athanasiou, et al. devices measure creep deformation and stress relaxation of the cartilage.

The prior art devices described above are relatively complex tools, having electronically driven vibrators or linear actuators, and are relatively expensive to manufacture. Hence, there is a need for a simple, portable, and effective tool ihat is easy to operate and inexpensive to manufacture.

### SUMMARY OF THE IRVENTION

The present invention is a simple, mechanical, hand-held probe that can be used in situ, in an open joint, or in an arthroscopic procedure to provide a surgeon with a quantitative assessment of the condition of the cartilage without the need for a vibratory or linear drive motor. The device also can be manufactured inexpensively, thereby making it amenable to use as a disposable, one-time use product.

In one aspect of the present invention, an apparatus for measuring the condition of an articular cartilage includes a housing and a probe having a proximal end disposed within the housing and a distal end adapted to contact a surface of the cartilage. The apparatus further includes a mass disposed within the housing at a position spaced from the proximal end of the probe. The mass is configured to deliver a predetermined force to the proximal end of the probe. The apparatus further includes an accelerator that is coupled to the mass and adapted to accelerate the mass to a predetermined velocity.

In another aspect of the present invention, art apparatus for measuring the condition of an articular cartilage includes a housing and a probe having a proximal end disposed within the housing and a distal end adapted to contact a surface of the cartilage. The apparatus further includes a mass disposed within the housing at a position spaced from the proximal end of the probe and adapted to deliver a predetermined force to the proximal end of the probe. The apparatus further includes an accelerator coupled to the mass, and an actuator coupled to the probe and adapted to actuate the accelerator upon placement of a predetermined displacement on the probe. The apparatus further includes a detector operatively connected to the probe that is adapted to detect a mechanical response of the cartilage.

In yet another aspect of the present invention, an apparatus for measuring the condition of an articular cartilage includes a housing having an internally disposed radial shoulder, and a probe having a proximal end disposed within the cartilage and a distal end that is extendable beyond a portion of the housing and adapted to contact a surface of the cartilage. The apparatus further includes a mass disposed within the housing at a position spaced from the proximal end of the probe and movable toward the proximal end of the probe. The apparatus further includes a compression spring disposed in the housing, a guide sleeve disposed within the housing, a guide stem having a portion disposed within the guide sleeve and an end connected to the mass, and an elongated rod having a first end operatively connected to the proximal end of the probe and a distal end having a latch adapted to controllably engage and release a portion of the guide stem attached to the mass. The apparatus further includes a load spring arranged to apply a predefined resistance load against movement of the distal end of the probe into the housing, a detector adapted to detect a mechanical response of the cartilage subsequent to impact of the mass with the proximal end of the probe, and a display device adapted to receive a signal from the detector and indicate the biomechanical condition of the cartilage.

In still another aspect of the present invention, a method for evaluating the condition of an articular cartilage includes contacting a surface of the cartilage with the distal end of a probe, and releasing a mass upon generation of a predetermined load on the probe. The method further includes accelerating the mass to a predetermined velocity to provide a predetermined force, and applying the predetermined force to a proximal (end of the probe, thereby transmitting the predetermined force through the distal end of the probe to the cartilage.

In an additional aspect of the present invention, a method for evaluating the condition of an articular cartilage includes contacting a surface of the articular cartilage with a distal end of a probe, and releasing a mass upon generation of a predefined load on the probe. The method further includes accelerating the mass to a predetermined velocity to create a predetermined force and applying the predetermined force to a proximal end of the probe, thereby transmitting the predetermined force through the distal end of the robe to the cartilage. The method further includes detecting a mechanical response of the cartilage subsequent to application of the predetermined force and assessing a biomechanical condition of the cartilage as a function of the mechanical response.

In one more aspect of the present invention, a method for quantitatively assessing the condition of an articular cartilage includes contacting the surface of the articular cartilage with a distal end of an axially displaceable probe, pressing the distal end of the axially displaceable probe against the surface of the cartilage whereby the probe is moved in an axial direction, and releasing a predefined mass in response to movement of the probe a predetermined axial distance while maintaining the distal end of the probe in pressed contact with the cartilage. The method further includes accelerating the mass to a predetermined velocity in a direction toward a proximal end of the probe and impacting the proximal end of the probe with the accelerated mass, thereby transmitting a predetermined force through the distal end of the probe to the cartilage. The method further includes detecting a mechanical response of the cartilage as a function of a measurement selected from the group consisting of depth of penetration of the probe, time course of the depth of penetration of the probe, acceleration of the probe, and probe rebound energy. The method further includes assessing a biomechanical condition of the cartilage as a function of the detected mechanical response.

### BRIEF DESCRIPTION OF THE DRAWINGS

A more complete understanding of the structure and operation of the present invention may be had by reference to the following detailed description when taken in conjunction with the accompanying drawings, wherein:

FIG. 1 is a quasi-schematic cross-sectional representation of the apparatus for measuring the condition of an articular cartilage in accordance with the present invention; and

FIG. 2 is a graphical representation of displacement with respect to time of the distal end of the probe illustrated in FIG. 1, when used in accordance with the method for measuring the condition of an articular cartilage in accordance with the present invention.

### DETAILED DESCRIPTION OF A PRESENTLY PREFERRED EXEMPLARY EMBODIMENT

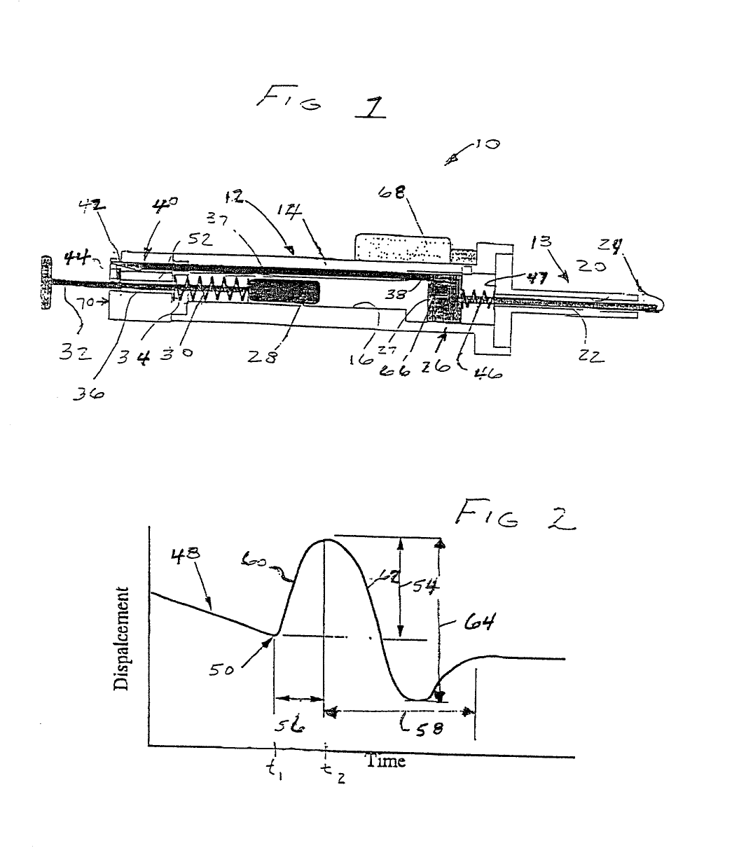

In a preferred embodiment of the present invention, an apparatus for measuring the condition of an articular cartilage is generally indicated in FIG. 1 by the reference numeral 10. Apparatus 10 has an outer housing 12 preferably formed of a relatively rigid material, such as metal or plastic. Housing 12 includes a main body portion 14, which has an internally disposed hollow chamber 16, and an outwardly extending distal portion 18, which has a centrally disposed hollow guide sleeve 20 extending therethrough. Hollow guide sleeve 20 of distal end portion 18 is preferably in direct communication with hollow chamber 16 of body portion 14.

Apparatus 10 further includes a probe 22 having a distal end 24 that is adapted to contact a surface of a cartilage when used in carrying out method embodying present invention, and a proximal end 26 disposed within hollow chamber 16 of body portion 14. In a preferred embodiment of present invention, probe 22 has a circular cross-section and distal end 24 of probe 22 is rounded to provide a smooth contact surface with cartilage. Proximal end 26 of probe 22 has an enlarged head portion 27 that is adapted to receive an impact mass thereon.

Apparatus 10 further includes a mass 28 that is disposed within hollow chamber 16 of body portion 14 of housing 12 at a position spaced from proximal end 26 of probe 22. Mass 28 is sized and configured to deliver, upon release, a predetermined force to proximal end 26 of probe 22. An accelerator 30, for example a compression spring as illustrated in an exemplary embodiment in FIG. 1, is coupled to mass 28 so that upon release, mass 28 is accelerated to a predetermined velocity at time of contact with head portion 27 of proximal end 26 of probe 22. In a preferred embodiment, spring 30 is disposed about an elongated stem 32 extending from mass 28, and when in its set position, i.e., position prior to release of mass 28, spring 30 is in a compressed state between an upper end of mass 28 mad an internally disposed radial shoulder 34 of housing 12. Guide stem 32 has a lower end connected to mass 28 and an upper portion that is partially disposed within a guide sleeve 36 provided in housing 12.

In a preferred embodiment of present invention, trigger mechanism by which mass 28 is released from its set position, as illustrated in FIG. 1, includes an elongated rod 37 having a first end 38 operatively connected to proximal end 26 of probe 22 and a distal end 40 having a latch 42 adapted to controllably engage and release a tab 44 extending outwardly from an upper portion of guide stem 32 attached to mass 28. Thus, in a preferred embodiment, trigger mechanism comprises a mechanical linkage between probe 22 and accelerator 30.

A load spring 46 is operatively connected between to enlarged head portion 27 of proximal end 26 of probe 22 and an internal radial shoulder 47 of distal end 18 of housing 12. Load spring 46 provides a predefined resistance force to provide for extension of distal end 24 of probe 22 beyond end of hollow guide sleeve 20 and application of a predefined resistance load during movement of distal end 24 of probe 22 into distal end 18 of housing 12. If desired, distal end 18 of apparatus 10 can easily be configured to be used at various approach angles to surface of cartilage, as illustrated in above-referenced earlier patents granted to Athanasiou, et al.

In operation, apparatus 10 is positioned over an articular cartilage to be tested. Distal end 24 of probe 22, for example a rod having a diameter of 3.1 mm, is brought into, and maintained in, contact with cartilage surface by the user of apparatus 10. Probe 22 is then slowly pressed towards cartilage surface by the user, during which time distal end 24 of probe 22 moves inwardly towards upper end 70 of apparatus 10, thereby producing a negative displacement as represented by line portion 48 of graph illustrated in FIG. 2. Pressure on and movement of probe 22 toward the cartilage surface is maintained by the user until a predetermined trigger load or displacement is reached, as represented by deflection (trigger load:) point 50 of graph shown in FIG. 2. Trigger load point 50 is point at which the load on distal end of probe is sufficient to move distal end 40 of elongated rod 37, which is connected to proximal end 26 of probe 22, through a distance sufficient to push latch 42 at distal end 40 of rod 36 past the tab 44 that extends outwardly from guide stem 32. This action pushes tab 44 downwardly, as viewed in FIG. 1, and permits tab 44 to slide along an internal slot 52 provided in upper end of housing 12. At that instant, mass 28 is released and is accelerated toward the distal end of the apparatus by spring 30, thereby applying a known kinetic energy to probe 22 upon contact with proximal end 26 of probe 22. The kinetic energy is directly transferred to distal end 24 of probe 22, which, in turn, loads the cartilage tissue at a high speed.

Kinetic energy is thus applied to the cartilage by impacting proximal end of probe 22 with a known mass 28 at a known velocity, thereby delivering a predetermined load. More specifically, impact mass 28, upon release latch 42, is accelerated to a predetermined velocity by compression spring 30. The physical response of- the cartilage tissue can be determined by measuring the depth of penetration, as represented by line portion 54 of graph illustrated in FIG. 2. The time of penetration (t₂-t₁) is represented by portion of graph indicated by reference numeral 56; the time of energy dissipation is represented by line portion 58, the respective acceleration and deceleration values are indicated by the slopes of line portions 60, 62, respectively, and probe rebound, i.e., the return energy from the tissue, is represented by graph portion 64.

In a preferred embodiment of the present invention, a motion-sensitive transducer 66, for example an accelerometer, is disposed in a portion of proximal end 26 of probe 22 and is operatively connected to a conventional integrated circuit 68 that is adapted to receive and display a signal provided by displacement transducer 66. Alternatively, integrated circuit 68 may be a unit that is physically separated from housing 12, but in electrical communication with transducer 66, or be replaced by a computer adapted to receive signals from displacement transducer 66 and provide a graphical and/or digital output of measured motion characteristics. Moreover, displacement and time data can be stored on a computer in addition to visually displaying data provided by integrated circuit 66. Other forms of motion detection with respect to time, either mounted on wall of housing 12 or on probe 22 itself can be used. Whichever form of detector is used, it should be operatively connected, either electrically, mechanically, magnetically or otherwise coupled, to probe 22 in such a manner as to detect a mechanical response of the cartilage and provide a signal indicative of the mechanical response subsequent to delivery of the predetermined force to the proximal end of probe. If so desired, detector 66 operatively connected to probe 22 may be employed to detect a mechanical response of cartilage prior to delivery of predetermined force to proximal end 26 of probe 22 i.e. slope/time characteristics of line portion 48 of the graph displayed in FIG. 2.

Integrated circuit 68 also may be considered as performing function of a correlator when adapted to correlate mechanical response with a biomechanical condition of cartilage. When a graphical representation is displayed, accelerations and energy absorption can be calculated directly from the graph. In actual experiments, time of impact ranged from about 1 to about 4 ms and penetration depth ranged from about 20 to about 200 μm. This data can be readily correlated to cartilage tissue's permeability, structural stiffness, structural recovery, and other biomechanical properties.

In another embodiment of present invention, a method for evaluating condition of an articular cartilage includes contacting a surface of cartilage with distal end 24 of probe 22 and releasing mass 28 upon generation of a predefined load or displacement on probe 22. As described above, the method further includes accelerating mass 28 to a predetermined velocity to create a predetermined force and then applying predetermined force to a proximal end 26 of probe 22, thereby transmitting the predetermined force through distal end 24 of probe 22 to the cartilage. Moreover, the method includes detecting the mechanical response of the cartilage to the predetermined force imposed by distal end 24 of probe 22 prior and/or subsequent to application of predetermined force. More specifically, the method may include detecting the mechanical response of the cartilage as a function of a depth measurement, such as depth of penetration of probe, a time course (i.e., an elapsed time period) for penetration of probe, acceleration of probe, deceleration of probe, and probe rebound energy.

Furthermore, the method may further include assessing the biomechanical condition of the cartilage as a function of the above-described mechanical responses, including wherein the assessed biomechanical conditions include permeability of articular cartilage. Since distal end 40 of elongated rod 36 is moved a predetermined distance upon application of a predetermined load on distal end 24 of probe 22, release of accelerator 30 and subsequent impact of mass 28 on proximal end 26 of probe 22, may be considered as being initiated by either application of a predetermined load on probe 22 provided by cartilage, or movement of probe 22 a predetermined distance as a result of pressing distal end 24 of probe 22 against surface of cartilage.

In actual tests, a correlation was established between time of impact and tissue permeability. more permeable tissue specimens took more time to reach maximum probe penetration. Therefore, in one testing mode it may be desirable to measure probe displacement and time prior to application of the low impact load on the probe.

Industrial Applicability

Apparatus 10 and the method for evaluating condition of an articular cartilage embodied by present invention is particularly useful in accurately assessing functional capability of articular cartilage to determine correct treatment modality whether, for example, hemiarthroplasty, total joint replacement, cartilage transplantation, regeneration, or other procedure is indicated. Surgeons need a tool to measure biomechanical properties of cartilage in situ. The property most descriptive and predictive of tissue's functional capability is deformation under load. Because they allow measurement of the cartilage's deformation under load, the method and apparatus embodying present invention, provide a simple, expedient, and accurate measure of mechanical condition of articular cartilage.

Moreover, in addition to being used in situ, apparatus and method can be used in an open joint or in an arthroscopic procedure to provide a surgeon with a quantitative assessment of the condition of the cartilage. Importantly, apparatuses embodying the present invention can be manufactured inexpensively, which makes the present invention readily amenable to being used as a disposable, one-time use product.

The present invention utilizes fast, controlled, post-contact loading applied onto a cartilage surface to determine the mechanical condition of the tissue. The apparatus employs a probe structure, for example, a rod, ball, or beam, that is placed and maintained in contact with cartilage surface during measurement of the condition of the cartilage. The probe is slowly pressed onto cartilage surface by user until a predetermined trigger load is reached. At that point, a known kinetic energy is applied to the proximal end of the probe, which in turn loads the cartilage at a high speed. Kinetic energy is applied by impacting the proximal end of the probe with a known mass at a known velocity. The physical response of the tissue is then determined by measuring at least one physical response, such as depth of penetration, time of energy dissipation, acceleration of the probe, and probe rebound or return energy, from the tissue. The measured data can then be correlated to the tissue's permeability, structural stiffness, and structural recovery. In summary, the apparatus 10 embodying the present invention applies a known kinetic energy, through impact, onto the surface of the cartilage, and then measures the physical response of the tissue. Advantageously, the device can be configured to be used at various approach angles to the surface of the cartilage.

Although the present invention is described in terms of a preferred embodiment, with a specific exemplary construction and method of using the apparatus 10, those skilled in the art will recognize that changes in that specific construction, for example in the latch mechanism or accelerator design, and changes in the specific exemplary method, may be made without departing from the spirit of the invention. Such changes are intended to fall within the scope of the following claims. Other aspects, features, and advantages of the present invention may be obtained from a study of this disclosure and the drawings, along with the appended claims.

What we claim is:

1. An apparatus for measuring the condition of a cartilage, comprising:
a housing;
a probe having a distal end adapted to contact a surface of the cartilage and a proximal end disposed within the housing;
a mass disposed within the housing at a position spaced from and configured to deliver a predetermined kinetic energy to said proximal end of said probe; and
an accelerator coupled to the mass and configured to accelerate the mass to a predetermined velocity.

2. The apparatus of claim 1, further comprising an actuator coupled to the probe and configured to actuate the accelerator upon placement of a predefined load on the probe.

3. The apparatus of claim 1, further comprising an actuator coupled to the probe and configured to actuate the accelerator upon displacement of the probe through a predetermined distance.

4. The apparatus of claim 1, further including a detector operatively coupled to the probe and adapted to detect a mechanical response of the cartilage.

5. The apparatus of claim 4, wherein the mechanical response is detected prior to delivery of the predetermined kinetic energy to the proximal end of the probe.

6. The apparatus of claim 4, further comprising a display device configured to display data representative of the detected mechanical response.

7. The apparatus of claim 4, wherein the mechanical response is detected subsequent to delivery of the predetermined kinetic energy to the proximal end of the probe.

8. The apparatus of claim 1, wherein the distal end of the probe extends beyond a portion of the housing.

9. The apparatus of claim 1, wherein the accelerator comprises a spring.

10. The apparatus of claim 1, wherein the actuator comprises a mechanical linkage between the probe and the accelerator.

11. The apparatus of claim 10, wherein the mechanical linkage comprises an elongated rod.

12. The apparatus of claim 1, further comprising a transducer operatively coupled to the probe.

13. The apparatus of claim 12, wherein the transducer comprises a digital transducer.

14. The apparatus of claim 1, further comprising a correlator coupled to the detector and adapted to correlate the mechanical response with a biomechanical condition of the cartilage.

15. The apparatus of claim 14, further comprising a display coupled to the housing and configured to show the biomechanical condition.

16. The apparatus, as set forth in claim 14, wherein the correlator comprises an integrated circuit.

17. An apparatus for measuring the condition of an articular cartilage, comprising:
a housing;
a probe having a distal end adapted to contact a surface of the cartilage and a proximal end disposed within the housing;
a mass disposed within the housing at a position spaced from and configured to deliver a predetermined force to the proximal end of the probe;
an accelerator coupled to the mass and configured to accelerate the mass to a predetermined velocity;
an actuator coupled to the probe and configured to actuate the accelerator upon placement of a predefined displacement on the probe; and
a detector operatively coupled to the probe and adapted to detect a mechanical response of the cartilage.

18. The apparatus of claim 17, further comprising a resistance load device operatively coupled to the probe.

19. An apparatus for measuring the condition of an articular cartilage, comprising:
a housing having an internally disposed radial shoulder;
a probe having a distal end extendable beyond a portion of the housing and adapted to contact a surface of the cartilage, and a proximal end disposed within the housing;
a mass disposed within the housing at a position spaced from the proximal end of the probe and movable in a direction toward the proximal end of the probe;
a compression spring disposed in the housing at a position between the internally disposed radial shoulder of the housing and the mass;
a guide sleeve disposed in the housing;
a guide stem having a portion disposed within the guide sleeve and an end connected to the mass;
an elongated rod having a first end operatively connected to the proximal end of the probe and a distal end having a latch adapted to controllably engage and release a portion of the guide stem attached to the mass;
a load spring arranged to apply a predefined resistance load against movement of the distal end of the probe into the housing;
a detector adapted to detect a mechanical response of the cartilage subsequent to impact of the mass with the proximal end of the probe and provide a signal correlative of the mechanical response; and
a display device adapted to receive the signal from the detector and display data indicative of the biomechanical condition of the cartilage.

20. A method for evaluating the condition of a cartilage, comprising:
contacting a surface of the cartilage with a distal end of a probe;
releasing a mass upon generation of a predefined load on the probe;
accelerating the mass to a predetermined velocity to create a predetermined kinetic energy; and
applying the predetermined kinetic energy to a proximal end of the probe thereby transmitting the predetermined kinetic energy through the distal end of the probe to the cartilage.

21. The method of claim 20, further including detecting a mechanical response of the cartilage.

22. The method of claim 21, wherein the mechanical response is detected prior to the application of the predetermined kinetic energy.

23. The method of claim 21, wherein the mechanical response is detected subsequent to the application of the predetermined kinetic energy.

24. The method of claim 23, wherein the method includes detecting the mechanical response of the cartilage as a function of a measured parameter selected from the group consisting of depth of penetration of the probe, time course of the depth of penetration of the probe, acceleration of the probe, and probe rebound energy.

25. The method of claim 21, further comprising assessing a biomechanical condition of the cartilage as a function of the mechanical response.

26. The method of claim 25, wherein the biomechanical condition includes permeability of the cartilage.

27. The method of claim 20, further comprising generating a predetermined load on the probe by pressing the distal end of the probe against the surface of the cartilage.

28. A method for evaluating the condition of an articular cartilage, comprising:
contacting a surface of the articular cartilage with a distal end of a probe;
releasing a mass upon generation of a predefined load on the probe;
accelerating the mass to a predetermined velocity to create a predetermined kinetic energy;
applying the predetermined force to a proximal end of the probe, thereby transmitting the predetermined kinetic energy through the distal end to the cartilage;
detecting a mechanical response of the cartilage subsequent to application of the predetermined kinetic energy; and
assessing a biomechanical condition of the cartilage as a function of the mechanical response.

29. The method of claim 28, further comprising detecting a mechanical response of the cartilage prior to the application of the predetermined kinetic energy.

30. The method of claim 29, wherein the method includes detecting the mechanical response of the cartilage as a function of a measured parameter selected from the group consisting of depth of penetration of the probe, time course of the depth of penetration of the probe, and acceleration of the probe.

31. A method for quantitatively assessing the condition of articular cartilage, comprising:
contacting the surface of the articular cartilage with the distal end of an axially displaceable probe;
pressing the distal end of the axially displaceable probe against the surface of the cartilage whereby the probe is moved in an axial direction;
releasing the predefined mass in response to movement of the probe a predetermined axial distance while maintaining the distal end of the probe in pressed contact with the cartilage;
accelerating the mass to a predetermined velocity in a direction toward a proximal end of the probe;
impacting the proximal end of the probe with the accelerated mass, thereby transmitting a predetermined kinetic energy through the distal end of the probe to the cartilage;
detecting a mechanical response of the cartilage as a function of a measured parameter selected from the group consisting of a depth of penetration of the probe, a time course of the depth of penetration of the probe, an acceleration of the probe, and a probe rebound energy; and
assessing a biomechanical condition of the cartilage as a function of the mechanical response.
